(19) United States
(12) Patent Application Publication
(10) Pub. No.: US 2019/0365738 A1
(43) Pub. Date: Dec. 5, 2019

(54) AMORPHOUS SOLID DISPERSION OF VALBENAZINE TOSYLATE AND PROCESS FOR PREPARATION THEREOF

(71) Applicant: CADILA HEALTHCARE LIMITED, Ahmedabad (IN)

(72) Inventors: Sanjay Jagdish DESAI, Ahmedabad (IN); Kuldeep Natwarlal JAIN, Ahmedabad (IN); Jitesh Amratlal DESAI, Ahmedabad (IN); Akshay Dineshchandra DESAI, Ahmedabad (IN)

(21) Appl. No.: 16/413,741

(22) Filed: May 16, 2019

(30) Foreign Application Priority Data
May 21, 2018 (IN) 201821018884

(51) Int. Cl. A61K 31/4745; A61K 47/32; A61K 47/38

(52) U.S. Cl. CPC A61K 31/4745; A61K 47/38; A61K 47/32

(57) ABSTRACT

There is disclosed an amorphous solid dispersion of valbenazine tosylate, and a process for preparation. There is also disclosed a pharmaceutical composition comprising amorphous solid dispersion of valbenazine tosylate:

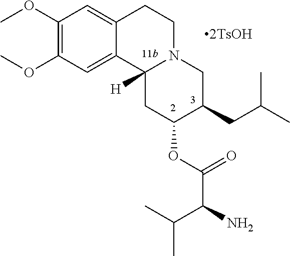 [SMILES: COc1cc2c(cc1OC)[C@H]1C[C@@H](OC(=O)[C@@H](N)C(C)C)[C@H](CC(C)C)CN1CC2]

### FIELD OF THE INVENTION

The present invention relates to an amorphous solid dispersion of valbenazine tosylate. In particular, the present invention relates to an amorphous solid dispersion of valbenazine tosylate and their process for preparation. The present invention also relates to a pharmaceutical composition comprising amorphous solid dispersion of valbenazine tosylate.

### BACKGROUND AND THE PRIOR ART

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

INGREZZA® is a vesicular monoamine transporter 2 (VMAT2) inhibitor indicated for the treatment of tardive dyskinesia. The active ingredient of the approved product INGREZZA® valbenazine tosylate which is described chemically as (2R,3R,11bR)-3-Isobutyl-9,10-dimethoxy-1,3,4,6,7,11b-hexahydro-2H-pyrido[2,1-a]isoquinolin-2-yl L-valinate ditosylate salt, is having the structure of Formula (I)

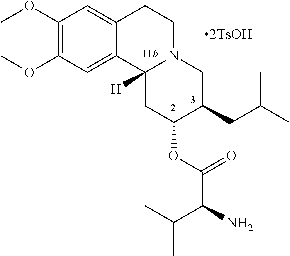 [SMILES: COc1cc2c(cc1OC)[C@H]1C[C@@H](OC(=O)[C@@H](N)C(C)C)[C@H](CC(C)C)CN1CC2]

U.S. Pat. No. 8,039,627 B2 discloses the compound valbenazine and provides its process for preparation.

U.S. Pat. No. 8,357,697 B2 relates to method of treating hyperkinetic disorder with a pharmaceutical composition comprising valbenazine tosylate.

US 20170145008 A1 discloses various crystalline forms of valbenazine tosylate designated as Form-I, II, III, IV, V, VI and amorphous form of valbenazine tosylate and process for preparing thereof.

US 20170145008 A1 also discloses crystalline Form-I and Form-II of valbenazine dihydrochloride and process for preparing thereof.

US 20170183346 A1 relates to process for preparing valbenazine tosylate.

WO 2018067945A1 discloses various crystalline forms of valbenazine tosylate or solvates designated as Form-T1 to T10, Form-T2 and valbenazine free base designated as Form L1 to L4 and process preparing thereof.

The prior-arts disclose one or the other crystalline forms of valbenazine tosylate or solvates or an amorphous form of valbenazine tosylate. There is no disclosure of amorphous solid dispersion of valbenazine tosylate in the art.

In view of the above art, there is provided an amorphous solid dispersion of valbenazine tosylate together with one or more pharmaceutically acceptable carrier and process for preparing thereof.

### SUMMARY OF THE INVENTION

In one general aspect, there is provided an amorphous solid dispersion of valbenazine tosylate of Formula (I)

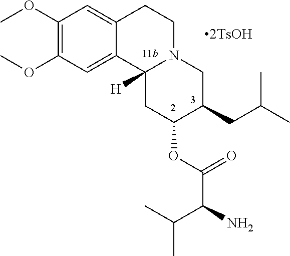 [SMILES: COc1cc2c(cc1OC)[C@H]1C[C@@H](OC(=O)[C@@H](N)C(C)C)[C@H](CC(C)C)CN1CC2]

together with one or more pharmaceutically acceptable carriers.

In another general aspect, there is provided a process for the preparation of an amorphous solid dispersion of valbenazine tosylate of Formula (I) together with one or to more pharmaceutically acceptable carriers.

In another general aspect, there is provided a process for the preparation of an amorphous solid dispersion of valbenazine tosylate of Formula (I) together with one or more pharmaceutically acceptable carriers, using spray drying.

In another general aspect, there is provided a process for the preparation of an amorphous solid dispersion of valbenazine tosylate together with one or more pharmaceutically acceptable carriers, the process comprising:

(a) providing a solution or suspension of valbenazine tosylate in presence of one or more carriers or excipients or polymers in one or more solvents; and
(b) obtaining the amorphous solid dispersion of valbenazine tosylate together with one or more pharmaceutically acceptable carriers by the removal of the solvents.

In another general aspect, there is provided a pharmaceutical composition containing an amorphous solid dispersion of valbenazine tosylate together with one or more pharmaceutically acceptable carriers.

In another general aspect, there is provided a pharmaceutical composition comprising an amorphous solid dispersion of valbenazine tosylate together with one or more pharmaceutically acceptable carriers, excipients or diluents.

### BRIEF DESCRIPTION OF THE ACCOMPANYING DRAWINGS

FIG. 1: x-ray powder diffractogram (XRPD) of amorphous solid dispersion of valbenazine tosylate with co-povidone as per Example-1.

FIG. 2: x-ray powder diffractogram (XRPD) of amorphous solid dispersion of valbenazine tosylate with hydroxy propyl methyl cellulose (HPMC) as per Example-2.

### DETAILED DESCRIPTION OF THE INVENTION

The aforementioned objectives of the present invention are fulfilled by one or more of the processes described herein.

All ranges recited herein include the endpoints, including those that recite a range “between” two values. Terms such as “about”, “from”, and “to” are to be construed as modifying a term or value such that it is not an absolute. This includes, at very least, the degree of expected experimental error, technique error and instrument error for a given technique used to measure a value.

As used herein, the term “solution” does not limit to a clear solution only and includes a hazy solution or slurry which is a heterogeneous mixture.

The term “pharmaceutical compositions” herein includes pharmaceutical formulations like tablets, pills, powders, liquids, suspensions, emulsions, granules, capsules, suppositories, or injection preparations.

The term “composition” used herein means a physical mixture of two or more components.

As used herein, the terms “obtaining” means isolating the amorphous solid dispersion of valbenazine tosylate by way of filtration, filtration under vacuum, centrifugation, and decantation. The product obtained may be further or additionally dried to achieve the desired moisture values. For example, the product may be dried in a tray drier, dried under vacuum and/or in a Fluid Bed Drier.

As used herein, the term “solid dispersion” means any solid composition having at least two components. In certain embodiments, a solid dispersion as disclosed herein includes an active ingredient valbenazine tosylate thereof dispersed among at least one or more pharmaceutically acceptable carriers or excipients.

The pharmaceutically acceptable carriers or excipients may be selected from polymers. Polymers may polyvinylpyrrolidone, hydroxypropyl methylcellulose, hydroxypropyl cellulose or hydroxypropyl methylcellulose acetate succinate, hydroxypropyl methylcellulose phthalate, cellulose acetate phthalate and polymethacrylate.

In one general aspect there is provided an amorphous solid dispersion of valbenazine tosylate of Formula (I),

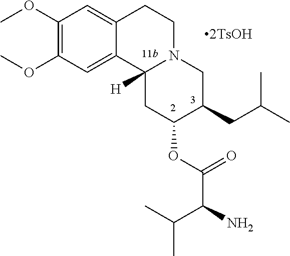 [SMILES: COc1cc2c(cc1OC)[C@H]1C[C@@H](OC(=O)[C@@H](N)C(C)C)[C@H](CC(C)C)CN1CC2]

with one or more pharmaceutically acceptable carriers.

In another general aspect, there is provided an amorphous solid dispersion of valbenazine tosylate with one or more pharmaceutically acceptable carriers having purity by HPLC of greater than about 99%.

In general, the amorphous solid dispersion of valbenazine tosylate with one or more pharmaceutically acceptable carriers is having residual solvents within the permissible ICH limits suitable for pharmaceutical preparations.

The amorphous solid dispersion of valbenazine tosylate with one or more pharmaceutically acceptable carriers, of the present invention is having water content not more than about 6% wt/wt.

In another general aspect, there is provided a process for the preparation of an amorphous solid dispersion of valbenazine tosylate with one or more pharmaceutically acceptable carriers, the process comprising:

(a) providing a solution or suspension of valbenazine tosylate and one or more pharmaceutically acceptable excipients in one or more solvents; and
(b) obtaining the amorphous solid dispersion of valbenazine tosylate by removal of the solvents.

The step (a) above involves providing a solution or suspension of valbenazine tosylate in one or more of solvents or mixture thereof.

The solution or suspension for step (a) can be obtained by known methods that include;

(i) direct use of a reaction mixture containing valbenazine tosylate that is obtained in the course of its synthesis; or
(ii) dissolving valbenazine tosylate in one or more of solvents or mixture thereof.

In general, in step (a) any physical form of valbenazine tosylate may be utilized for providing the solution of valbenazine tosylate in one or more of solvents or mixture thereof. The dissolution temperatures may be from about below 0° C. to about the reflux temperature of the solvent.

In general, the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In general, the CJ-4 alcohol is selected from methanol, ethanol, n-propanol, isopropanol and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone or mixture thereof.

In general, the amorphous solid dispersion of valbenazine tosylate having a purity by HPLC of more than about 99%.

In general, the pharmaceutically acceptable carrier may be selected from polymers. Polymers may be selected from methacrylic acid copolymers; polyvinylpyrrolidone (PVP), 4-vinylpyrrolidone-vinyl acetate copolymer (copovidone) or copolymers of methacrylic acid and ethylacrylate (EUDRAGIT®, L100-55), hydroxy-propyl cellulose, hydroxypropylmethyl cellulose (HPMC), polymethylacrylate, hypromellose phthalate, or hydroxypropylmethyl cellulose acetate succinate (HPMC-AS). In particular, PVP of different grades like K-15, K-30, K-60, K-90 and K-120 may be used for the preparation of amorphous solid dispersion. More particular, hydroxypropylmethyl cellulose (HPMC) or hydroxypropylmethyl cellulose acetate succinate (HPMC-AS), 4-vinylpyrrolidone-vinyl acetate copolymer (copovidone) and PVP K-30 may be used.

In another general aspect, there is provided an amorphous solid dispersion comprising valbenazine or its pharmaceutically acceptable salts and one or more pharmaceutically acceptable excipients.

In some embodiments, the valbenazine tosylate of Formula (I) may be dispersed within a matrix formed by a polymer in its solid state such that it is immobilized in its amorphous form. The polymer may prevent intramolecular hydrogen bonding or weak dispersion forces between two or more drug molecules of valbenazine tosylate.

In some embodiments, the ratio of the amount of weight of valbenazine tosylate within the solid dispersion to the amount by weight of the polymer therein is from about 1:1 to about 1:10. The composition of valbenazine tosylate with polymer may be prepared by using about 1:1 to about 1:10 polymers with respect to valbenazine tosylate.

The step (b) above involves obtaining of an amorphous solid dispersion of valbenazine tosylate from the solution or suspension of step (a). The isolation of an amorphous solid dispersion of valbenazine tosylate may be affected by removing the solvents. The techniques which may be used for the removal of solvents comprises one or more of distillation, distillation under vacuum, spray drying, agitated thin film drying (“ATFD”), freeze drying (lyophilization), filtration, decantation, and centrifugation. The solvent may also be removed, optionally, at reduced pressure and/or at elevated temperature.

In another general aspect, there is provided a process for the preparation of an amorphous form of valbenazine tosylate, the process comprising:

(a) providing a solution or suspension of valbenazine tosylate in one or more solvents; and
(b) obtaining the amorphous solid dispersion of valbenazine tosylate by removal of the solvents.

The step (a) above involves providing a solution or suspension of valbenazine tosylate in one or more of solvents or mixture thereof.

The solution or suspension for step (a) can be obtained by known methods that include:

(i) direct use of a reaction mixture containing valbenazine tosylate that is obtained in the course of its synthesis; or
(ii) dissolving valbenazine tosylate in one or more of solvents or mixture thereof.

In general, in step (a) any physical form of valbenazine tosylate may be utilized for providing the solution of valbenazine tosylate in one or more of solvents or mixture thereof. The dissolution temperatures may be from about below 0° C. to about the reflux temperature of the solvent.

In general, the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In general, the C₁₋₄ alcohol is selected from methanol, ethanol, n-propanol, isopropanol and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone or mixture thereof.

In another general aspect, there is provided a process for preparation of amorphous solid dispersion of valbenazine tosylate with one or more pharmaceutically acceptable carriers by spray drying the solution of valbenazine tosylate in presence of excipient in one or more solvents.

The step (b) above involves obtaining of an amorphous solid dispersion of valbenazine tosylate with one or more pharmaceutically acceptable carriers from the solution or suspension of step (a). The isolation of an amorphous solid dispersion of valbenazine tosylate with one or more pharmaceutically acceptable carriers may be affected by removing the solvents. The techniques which may be used for the removal of solvents comprises one or more of distillation, distillation under vacuum, spray drying, agitated thin film drying (“ATFD”), freeze drying (lyophilization), filtration, decantation, and centrifugation. The solvent may also be removed, optionally, at reduced pressure and/or at elevated temperature.

In another general aspect, there is provided a process for preparation of an amorphous solid dispersion form of valbenazine tosylate with one or more pharmaceutically acceptable carriers, comprising spray drying a solution of valbenazine tosylate with a polymer. In general, the process involves spray drying of the feed stock.

In general, the preferred aspect of the invention involves spray drying of feed stock which is prepared as discussed herein below, wherein any solid forms of valbenazine tosylate may be used. In particular, the spray drying of valbenazine tosylate may be performed maintaining the inlet temperature in the range of 35° C.-80° C., nitrogen pressure of 2-6 kg/cm², maintaining the outlet temperature in the range of 30° C. to 60° C., at a feed rate of 15% to 20% and maintaining the vacuum at 30-120 mm of Hg using JISL Mini LSD-48 or LU-222 advanced model (twin cyclone) type spray driers.

Any known form of valbenazine tosylate or the filtered cake that is obtained as an end result of the reaction, or reaction mass comprising valbenazine tosylate or solution comprising valbenazine tosylate can be used as input for the preparation of feed stock.

In the present invention, feed stock of valbenazine tosylate of Formula (I) is conveniently prepared by dissolving valbenazine tosylate in one or more solvents. The solvents is selected from the group comprising one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In particular, methanol, ethanol, acetone, ethyl acetate, methylene dichloride, water-methanol, water-ethanol, water-acetone or mixture of solvents is used or such solvents that evaporate easily to afford dry product. Most particularly, acetone, methanol, ethanol, ethyl acetate, methylene dichloride or mixtures thereof may be used.

According to further general aspect, any form of valbenazine tosylate can be spray dried by dissolving or suspending or slurring in one or more solvents or solvent-water system to get amorphous form. In the present invention, feed stock of valbenazine tosylate in methanol is spray-dried. Thus obtain spry-dried compound is in amorphous form.

In another general aspect, there is provided a process for preparation of amorphous solid dispersion valbenazine tosylate of Formula (I) by temperature alterations of valbenazine tosylate of Formula (I) with a polymer in presence or absence of one or more solvents.

In another general aspect, there is provided a process for the preparation of an amorphous solid dispersion comprising valbenazine tosylate with one or more pharmaceutically acceptable carriers or excipients, the process comprising:

(a) providing a solution of valbenazine tosylate and one or more pharmaceutically acceptable carriers or excipients in one or more solvents; and
(b) obtaining the amorphous solid dispersion of valbenazine tosylate with one or more pharmaceutically acceptable carriers or excipients by the removal of the solvents.

In general, a solution of valbenazine tosylate in presence of one or more excipients in one or more solvents is obtained by the known methods that include:

(i) direct use of a reaction mixture containing valbenazine tosylate that is obtained in the course of its synthesis and addition of excipients; or
(ii) dissolving valbenazine tosylate and excipients in one or more solvents.

In general, in step a) any physical form of valbenazine tosylate may be utilized for providing the solution of valbenazine tosylate in one or more solvents. The dissolution temperatures may be from 0° C. to the reflux temperature of the solvent. In particular, the dissolution may be performed from 25° C. to 120° C., so as to obtain the clear solution of valbenazine tosylate.

In general, the solvent comprises one or more of C₁₋₄ alcohols, C₂₋₆ esters, ketones, halogenated hydrocarbons, polar aprotic solvents, tetrahydrofuran, 2-methyltetrahydrofuran, dioxane or mixtures thereof.

In general, the C₁₋₄ alcohol is selected from methanol, ethanol, n-propanol, isopropanol, and n-butanol; the C₂₋₆ ester is selected from ethyl acetate, propyl acetate, isopropyl acetate, t-butyl acetate, and isobutyl acetate; the ketone is selected from acetone, methyl ethyl ketone, and methyl isobutyl ketone; the halogenated hydrocarbon is selected from methylene dichloride, ethylene dichloride, carbon tetrachloride and chlorobenzene; and the polar aprotic solvent is selected from dimethylformamide, dimethylsulfoxide, and N-methylpyrrolidone or mixture thereof.

In general, the excipients comprises of non-ionic polymer or an ionic polymer. In particular, the polymer is selected from methacrylic acid copolymers, polyvinylpyrrolidone (PVP), 4-vinylpyrrolidone-vinyl acetate copolymer (copovidone) or copolymers of methacrylic acid and ethylacrylate (EUDRAGITR® L100-55), hydroxypropyl cellulose, hydroxypropylmethyl cellulose (HPMC), hypromellose phthalate, or hydroxypropylmethyl cellulose acetate succinate (HPMC-AS). In particular, PVP of different grades like K-15, K-30, K-60, K-90 and K-120 may be used to prepare the feed stock. More particular, hydroxypropylmethyl cellulose (HPMC) or its acetate succinate and PVP K-30 may be used. HPMC with viscosity 8 cps, 5 cps or 3 cps may be used.

In some embodiments, the ratio of the amount of weight valbenazine tosylate of Formula (I) and the amount by weight of the excipient is from about 1:1 to about 1:10. The solution of valbenazine tosylate in presence of excipient in one or more solvents, preferably PVP K-30 or HPMC-AS may be prepared by using about 1:1 to about 1:10 polymers with respect to valbenazine tosylate.

The step b) involves removal of the solvent to obtain an amorphous solid dispersion of valbenazine tosylate. The isolation may be affected by removing solvents. Techniques which may be used for the removal of solvents include distillation, distillation under vacuum, spray drying, agitated thin film drying (“ATFD”), and freeze drying (lyophilization).

The solvent may be removed, optionally under reduced pressures, at temperatures less than 70° C., less than 60° C., less than 50° C.

In general, freeze drying (lyophilization) may be performed by freezing a solution of valbenazine tosylate optionally in presence of one or more excipients at low temperatures and reducing the pressure to remove the solvents from the frozen solution of valbenazine tosylate. Temperatures that may be required to freeze the solution, depending on the solvents chosen to make the solution of valbenazine tosylate may range from −70° C. to 10° C.

In general, the preferred aspect of the invention involves spray drying of valbenazine tosylate solution comprises of spray drying of feed stock, which is prepared as discussed below, wherein any solid forms of valbenazine tosylate in presence of one or more excipients is used. In particular, the spray drying of valbenazine tosylate hydrates in presence of excipients may be performed maintaining the inlet temperature in the range of 35° C.-80° C. nitrogen pressure of 2-6 kg/cm², maintaining the outlet temperature in the range of 30° C. to 60° C., at a feed rate of 15% to 20% and maintaining the vacuum at 30-120 mm of Hg using JISL Mini LSD-48 or LU-222 advanced model (twin cyclone) type spray driers.

In another general aspect, the present invention provides an amorphous solid dispersion of valbenazine tosylate with one or more pharmaceutically acceptable carriers, having purity by HPLC of >99%. In particular, the purity by HPLC of >99.5%, more particularly, the purity by HPLC of >99.8%, most particularly, the purity by HPLC of >99.9%.

In another general aspect, the present invention provides an amorphous form of solid valbenazine tosylate with one or more pharmaceutically acceptable carriers having purity by HPLC of >99%. In particular, the purity by HPLC of >99.5%, more particularly, the purity by HPLC of >99.8%, most particularly, the purity by HPLC of >99.9%.

The invention also encompasses a pharmaceutical composition containing an amorphous form of valbenazine tosylate with one or more pharmaceutically acceptable carriers. As used herein, the term “pharmaceutical compositions” includes pharmaceutical formulations comprises one or more of tablets, pills, powders, liquids, suspensions, emulsions, granules, capsules, suppositories, and injection preparations.

In another general aspect, there is provided a pharmaceutical composition containing an amorphous form of valbenazine tosylate with one or more pharmaceutically acceptable carriers and one or more pharmaceutically acceptable excipients.

In another general aspect, there is provided a pharmaceutical composition comprising an amorphous solid dispersion containing valbenazine tosylate together with one or more pharmaceutically acceptable carriers, excipients or diluents.

In general, the pharmaceutical compositions containing the amorphous solid dispersion of valbenazine tosylate with one or more pharmaceutically acceptable carriers, of the invention may be prepared by using diluents or excipients selected from fillers, bulking agents, binders, wetting agents, disintegrating agents, surface active agents and lubricants.

Various modes of administration of the pharmaceutical compositions of the invention is selected depending on the therapeutic purpose, for example tablets, pills, powders, liquids, suspensions, emulsions, granules, capsules, suppositories, or injection preparations.

The present invention is further illustrated by following examples which are provided merely to exemplify the invention and do not limit the scope of it.

### EXAMPLES

### Example-1: Preparation of Amorphous Solid Dispersion of Valbenazine Tosylate of Formula (I)

Methanol (300 ml) and valbenazine tosylate of Formula (I) (10 g) were added at 25° C. to 35° C. The reaction mass was stirred for 15 to 20 minute to get a clear solution. Co-povidone VA-64 (10 g) was added to the reaction mass and stirred for 15 to 20 minute to get a clear solution. Solvent was distilled out below 40° C. and residue was degassed till solid obtained as amorphous dispersion of valbenazine tosylate of Formula (I).

### Example-2: Preparation of Amorphous Solid Dispersion of Valbenazine Tosylate of Formula (I)

Methanol (1000 ml) and valbenazine tosylate of Formula (I) (10 g) were added at 25° C. to 35° C. The reaction mass was stirred for 15 to 20 minute to get a clear solution. Hydroxy propyl methyl cellulose (HPMC) (10 g) was added to the reaction mass and stirred for 15 to 20 minute to get a clear solution. Solvent was distilled out below 40° C. and residue was degassed till solid obtained as amorphous dispersion of valbenazine tosylate of Formula (I).

We claim:

1. An amorphous solid dispersion of valbenazine tosylate of Formula (I), 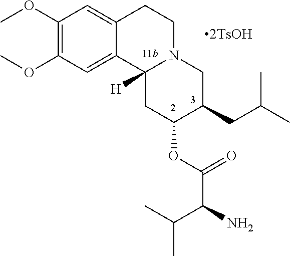 [SMILES: COc1cc2c(cc1OC)[C@H]1C[C@@H](OC(=O)[C@@H](N)C(C)C)[C@H](CC(C)C)CN1CC2]
together with one or more pharmaceutically acceptable carriers.

2. The amorphous solid dispersion of claim 1, wherein the pharmaceutically acceptable carrier is a polymer selected from the group consisting of methacrylic acid copolymers; polyvinylpyrrolidone (PVP), 4-vinylpyrrolidone-vinyl acetate copolymer (copovidone), or copolymers of methacrylic acid and ethylacrylate (EUDRAGIT® L100-55), hydroxypropyl cellulose, hydroxypropyl methyl cellulose (HPMC), polymethylacrylate, hypromellose phthalate, or hydroxypropyl methylcellulose acetate succinate (HPMC-AS).

3. The amorphous solid dispersion of claim 2, wherein the pharmaceutically acceptable carrier is selected from 4-vinylpyrrolidone-vinyl acetate copolymer (copovidone) and hydroxypropyl methyl cellulose (HPMC).

4. The amorphous solid dispersion of claim 2, wherein the ratio of valbenazine tosylate to polymer is about 1:1 to about 1:10.

5. The amorphous solid dispersion of claim 1, having a purity by HPLC greater than about 99.9%.

6. The amorphous solid dispersion of claim 1, having a water content not more than about 6% wt/wt.

7. An amorphous solid dispersion of valbenazine tosylate of Formula (I), 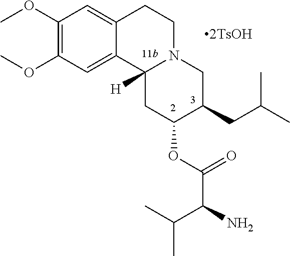 [SMILES: COc1cc2c(cc1OC)[C@H]1C[C@@H](OC(=O)[C@@H](N)C(C)C)[C@H](CC(C)C)CN1CC2]
with 4-vinylpyrrolidone-vinyl acetate copolymer (copovidone).

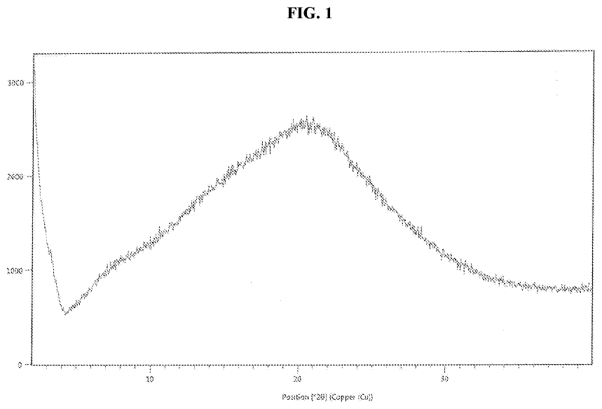

8. An amorphous solid dispersion of valbenazine tosylate of Formula (I), 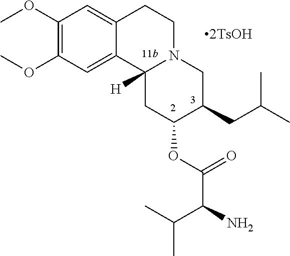 [SMILES: COc1cc2c(cc1OC)[C@H]1C[C@@H](OC(=O)[C@@H](N)C(C)C)[C@H](CC(C)C)CN1CC2]
with 4-vinylpyrrolidone-vinyl acetate copolymer (copovidone) having an X-ray diffraction pattern substantially as shown in FIG. 1.

9. An amorphous solid dispersion of valbenazine tosylate of Formula (I), 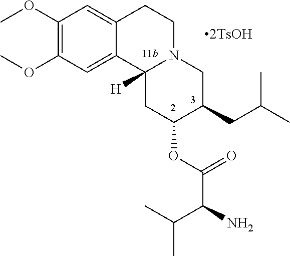 [SMILES: COc1cc2c(cc1OC)[C@H]1C[C@@H](OC(=O)[C@@H](N)C(C)C)[C@H](CC(C)C)CN1CC2]
with hydroxypropyl methyl cellulose (HPMC).

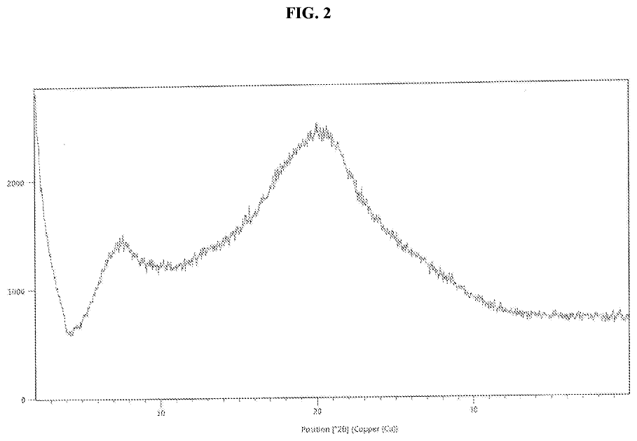

10. An amorphous solid dispersion of valbenazine tosylate of Formula (I), 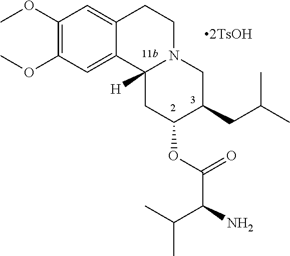 [SMILES: COc1cc2c(cc1OC)[C@H]1C[C@@H](OC(=O)[C@@H](N)C(C)C)[C@H](CC(C)C)CN1CC2]
with hydroxypropyl methyl cellulose (HPMC) having an X-ray diffraction pattern substantially as shown in FIG. 2.

11. A process for the preparation of an amorphous solid dispersion of valbenazine tosylate with one or more pharmaceutically acceptable carriers, the process comprising:
(a) providing a solution or suspension of valbenazine tosylate and one or more pharmaceutically acceptable excipients in one or more solvents; and
(b) obtaining the amorphous solid dispersion of valbenazine tosylate by removal of the solvents.

12. The process of claim 11, wherein the pharmaceutically acceptable carrier is selected from 4-vinylpyrrolidone-vinyl acetate copolymer (copovidone) and hydroxypropyl methyl cellulose (HPMC).

13. The process of claim 1, wherein the solvent is selected from C₁₋₄ alcohols, C₂₋₆ esters, ketones, halogenated hydrocarbons, polar aprotic solvents, tetrahydrofuran, 2-methyltetrahydrofuran, dioxane, or mixtures thereof.
